(19) United States
(12) Patent Application Publication
(10) Pub. No.: US 2008/0076928 A1
(43) Pub. Date: Mar. 27, 2008

(54) Novel pharmaceutical salts of 1-benzyl-4-&lsqb; (5,6-dimethoxy-1-indanone)-2-yl&rsqb; methyl piperidine ( Donepezil)

(76) Inventors: Venkatasubramanian Radhakrishnan Tarur, Mumbai (IN); Dhanajay Govind Sathe, Mumbai (IN); Avinash Vankatraman Naidu, Mumbai (IN); Kamiesh Digamber Sawant, Mumbai (IN); Tushar Anil Naik, Mumbai (IN); Umesh Parashram Aher, Mumbai (IN); Sachin Shivali Patil, Mumbai (IN)

Correspondence Address: PHARMACEUTICAL PATENT ATTORNEYS, LLC, 55 MADISON AVENUE, 4TH FLOOR, MORRISTOWN, NJ, 07960-7397, US

(21) Appl. No.: 11/412,294

(22) Filed: Apr. 27, 2006

Related U.S. Application Data
(63) Continuation-in-part of application No. 10/879,816, filed on Jun. 29, 2004.
Application No. 10/879,816 is a continuation-in-part of application No. 11/072,169, filed on Mar. 4, 2005.
Application No. 11/072,169 is a continuation-in-part of application No. 11/145,202, filed on Jun. 3, 2005, now Pat. No. 7,186,842.
(60) Provisional application No. 60/752,640, filed on Dec. 21, 2005.

(30) Foreign Application Priority Data
Jul. 28, 2004 (IN) PCT/IN04/00227

(51) Int. Cl. C07D 221/00

(52) U.S. Cl. 546/206

(57) ABSTRACT

New salts of donepezil, and new polymorphic forms of donepezil and its salts, and methods to prepare both the amorphous form and various polymorphic forms of donepezil or its salts.

### RELATED APPLICATIONS

This application is a continuation in part of co-pending application Ser. Nos. 11/072,169, filed 4 Mar. 2005; 11/145,202, filed 5 Jun. 2005; and 10/879,816, filed 29 Jun. 2004, the contents of which are incorporated by reference. This application further claims priority from application Ser. No. 60/752,640, filed 23 Dec. 2005, the contents of which are incorporated by reference.

### GOVERNMENT INTEREST

None.

### INTRODUCTION

The present invention relates to novel pharmaceutical salts of 1-benzyl-4-[(5,6-dimethoxy-1-indanone)-2-yl] methyl piperadine, commonly known as Donepezil, and shown as structure (I):

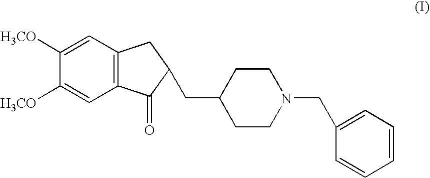

The process for the preparation of 1-benzyl-4-[(5,6-dimethoxy-1-indanone)-2-yl] methyl piperidine has been described in JP A 64-79151 (U.S. Pat. No. 4,895,841, EP 296560).

Japanese patent application, publication No. A-64-79151 (U.S. Pat. No. 4,895,841, EP 296560) discloses the hydrochloride salt of Donepezil, 1-benzyl-4-[(5,6-dimethoxy-1-indanone)-2-yl] methyl piperidine as in particular having an excellent action as a prophylactic and a therapeutic agent for senile dementia, and in particular as a prophylactic and a therapeutic agent for Alzheimer's disease and an industrial process for producing the same.

The compound Donepezil, {(1-benzyl-4-[(5,6-dimethoxy-1-indanone)-2-yl] methyl piperidine}, hereinafter is referred as compound 1.

It has been reported in our previously-published parent application, U.S. publication No. 2005/0107613, that compound (I) forms a novel oxalate salt, and exists in three polymorphic forms. We have also discovered that compound (I) forms novel maleate and fumarate salts. The novel salts can be prepared by an efficient, economic and reproducible process and particularly suited to large-scale preparation. The salts are therefore surprisingly amenable to large scale pharmaceutical processing and formulation.

The present invention specifically relates to the novel polymorphic form I of Donepezil maleate, form I Donepezil fumarate salts and the amorphous forms. The present invention also relates to the process for preparing amorphous form of Donepezil oxalate, characterized by powder X-ray diffraction and or infrared absorption peaks recorded in potassium bromide.

Polymorphic crystal of compound (I) maleate are hereafter referred as maleate form (I).

Polymorphic crystal of compound (I) fumarate are hereafter referred as fumarate form (I).

The maleate salt provides a melting point in the range of from 120 to 140° C.

The fumarate salt provides a melting point in the range of from 172 to 175° C.

The present invention encompasses the maleate and fumarate salts isolated in a purified form.

Also, the invention provides the maleate and fumarate salts in a pharmaceutically acceptable form, especially in bulk form, such form having good flow properties, especially good bulk flow properties.

The invention also provides a process for the preparation of the maleate salt, in which 1-benzyl-4-[(5,6-dimethoxy-1-indanone)-2-yl] methyl piperidine (compound I) is preferably dispersed or dissolved in a suitable solvent such as ethyl acetate and reacted with maleic acid in a solvent; and thereafter the maleate formed is recovered by filtration.

The solvent is an alkanol or a ketone selected from methanol, acetone and propan-2-ol.

The Donepezil maleate thus obtained is purified by dissolving in a suitable solvent and further recrystallized using an antisolvent.

A suitable solvent is selected from an alcohol, ether, ketone and water and the antisolvent is selected from ether, nitrile, water, alcohol, and a hydrocarbon.

The concentration of compound (I) is preferably in the range of from 3 to 25% weight/volume, more preferably in the range of from 5 to 20%. The concentration of maleic acid solution is preferably in the range of from 3 to 50% weight/volume.

The reaction is usually carried out at ambient temperature or at an elevated temperature, although any convenient temperature that provides the required product may be employed. A preferred temperature is in the range of 20-120° C.

The invention also provides a process for preparation of amorphous form of Donepezil maleate, which comprises of spray drying the solution of Donepezil maleate in suitable volatile organic solvents or by evaporating the solution under vacuum. The solvent selected is methylene dichloride. The spray drying is carried out at inlet temperature range of 120° C. to 200° C. and outlet temperature range of 60° C. to 110° C.

The invention also provides a process for the preparation of the fumarate salt, in which 1-benzyl-4-[(5,6-dimethoxy-1-indanone)-2-yl] methyl piperidine (compound I) is preferably dispersed or dissolved in a suitable solvent such as ethyl acetate and reacted with fumaric acid in a solvent; and thereafter the fumarate formed is recovered by filtration.

The solvent is an alkanol or a ketone selected from methanol, acetone and propan-2-ol.

The Donepezil fumarate thus obtained is purified by dissolving in a suitable solvent and further recrystallized using an antisolvent.

A suitable solvent is selected from an alkanol, ketone, water and ether while the antisolvent is selected from an ether, water, alkanol, hydrocarbon and a nitrile.

The concentration of compound (I) is preferably in the range of from 3 to 25% weight/volume, more preferably in the range of from 5 to 20%. The concentration of maleic acid solution is preferably in the range of from 3 to 50% weight/volume.

The reaction is usually carried out at ambient temperature or at an elevated temperature, although any convenient temperature that provides the required product may be employed. A preferred temperature is in the range of 20-120° C.

The invention also provides a process for preparation of amorphous form of Donepezil Fumarate, which comprises of spray drying the solution of Donepezil Fumarate in suitable volatile organic solvents or by evaporating the solution under vacuum. The solvent selected are, C₁-C₃ alcohols like methanol ethanol or isopropyl alcohol, preferably methanol. The spray drying is carried out at inlet temperature range of 120° C. to 200° C. and outlet temperature range of 60° C. to 110° C.

In addition to above two organic salts the present invention also provides a process to produce the amorphous form of Donepezil oxalate. The process comprises of spray drying the solution of Donepezil oxalate in suitable volatile organic solvents or in water or by evaporating the solution under vacuum. The solvent selected is methylene dichloride. The spray drying is carried out at inlet temperature range of 120° C. to 200° C. and outlet temperature range of 60° C. to 110° C.

### BRIEF DESCRIPTION OF FIGURES

FIG. 1 shows a powder X-ray diffraction pattern for Donepezil maleate form (I) crystals.

FIG. 2 shows a powder X-ray diffraction pattern for Donepezil fumarate form (I) crystals.

FIG. 3 shows a powder X-ray diffraction pattern for amorphous form of Donepezil maleate

FIG. 4 shows a powder X-ray diffraction pattern for amorphous form of Donepezil fumarate

FIG. 5 shows a powder X-ray diffraction pattern for amorphous form of Donepezil oxalate

FIG. 6 shows an infrared absorption spectrum for Donepezil maleate form (I) crystals

FIG. 7 shows an infrared absorption spectrum for Donepezil fumarate form (I) crystals

FIG. 8 shows an infrared absorption spectrum for amorphous form for Donepezil Maleate

FIG. 9 shows an infrared absorption spectrum for amorphous form for Donepezil fumarate

FIG. 10 shows an infrared absorption spectrum for amorphous form for Donepezil oxalate

(1) Maleate Form (I) Crystals

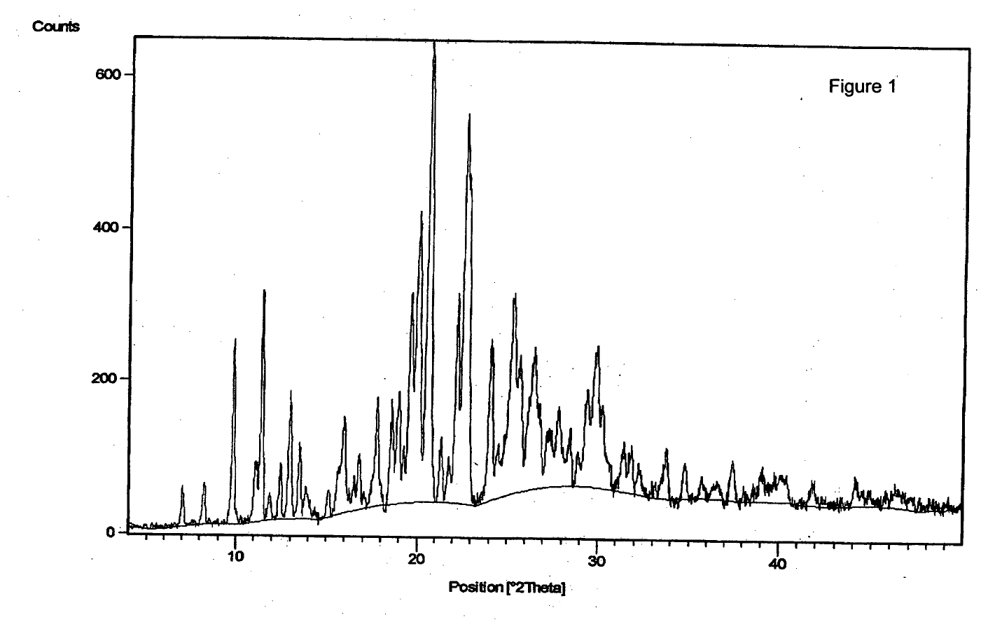

Peaks in powder X-ray diffraction pattern (See FIG. 1)

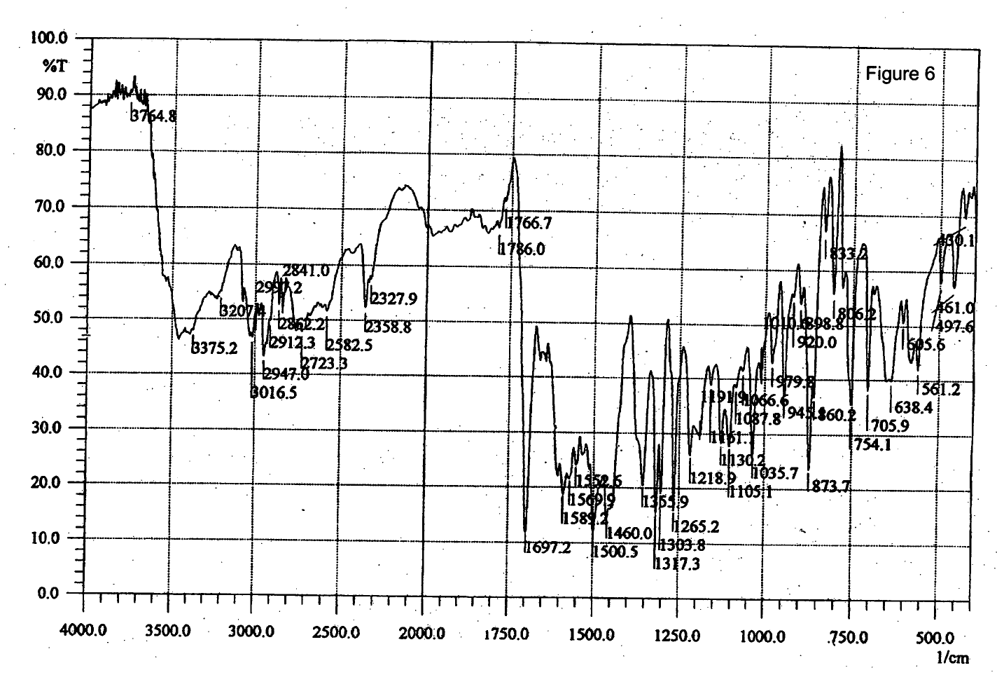

Peaks in infrared absorption spectrum recorded in potassium bromide (See FIG. 6)

(2) Amorphous Maleate Form

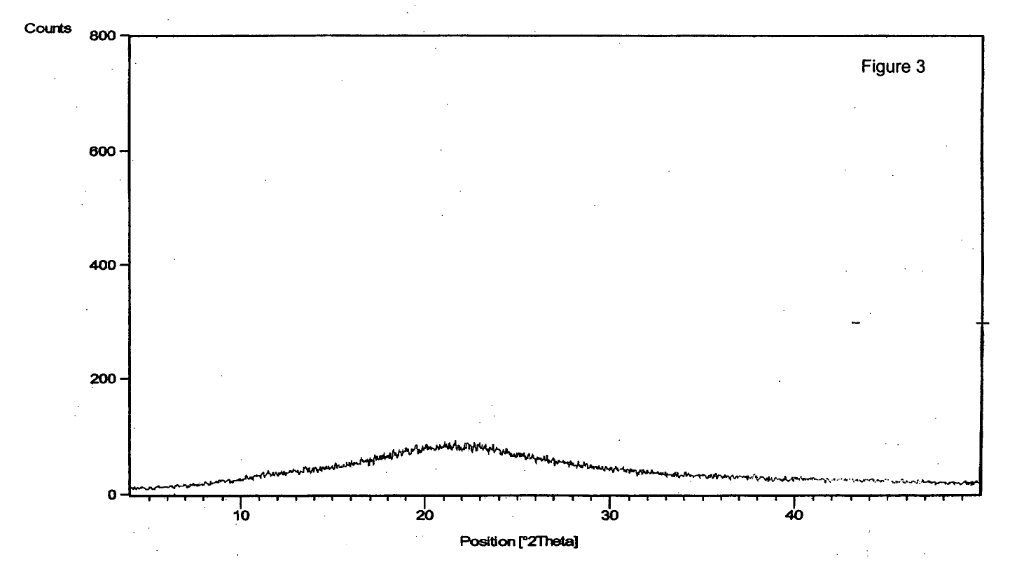

Peaks in powder X-ray diffraction pattern (See FIG. 3)

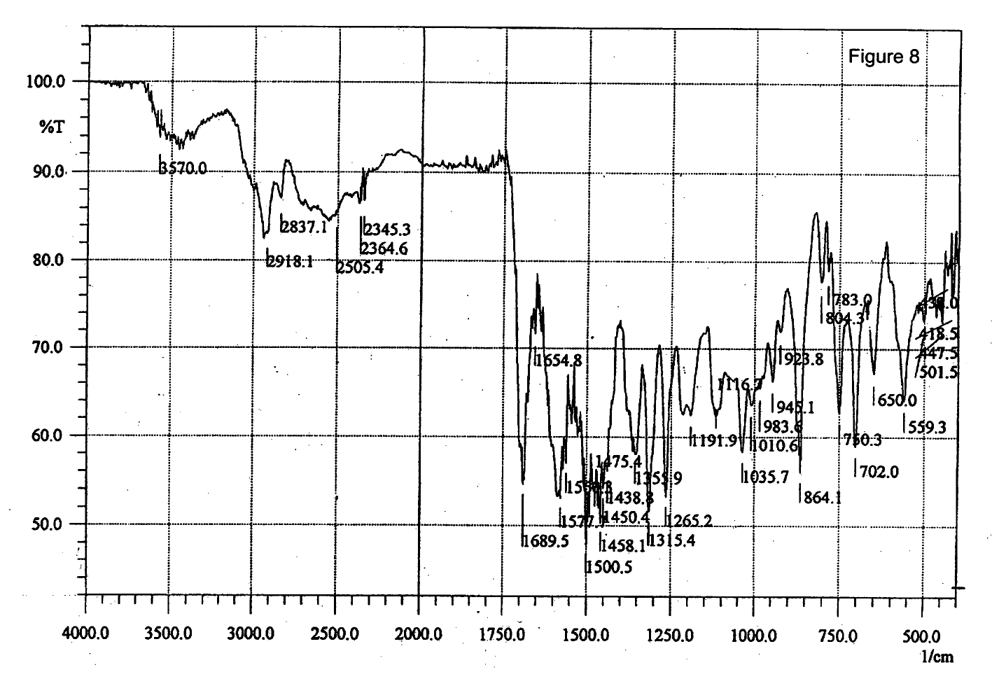

Peaks in infrared absorption spectrum recorded in potassium bromide (See FIG. 8)

(3) Fumarate Form (I) Crystals

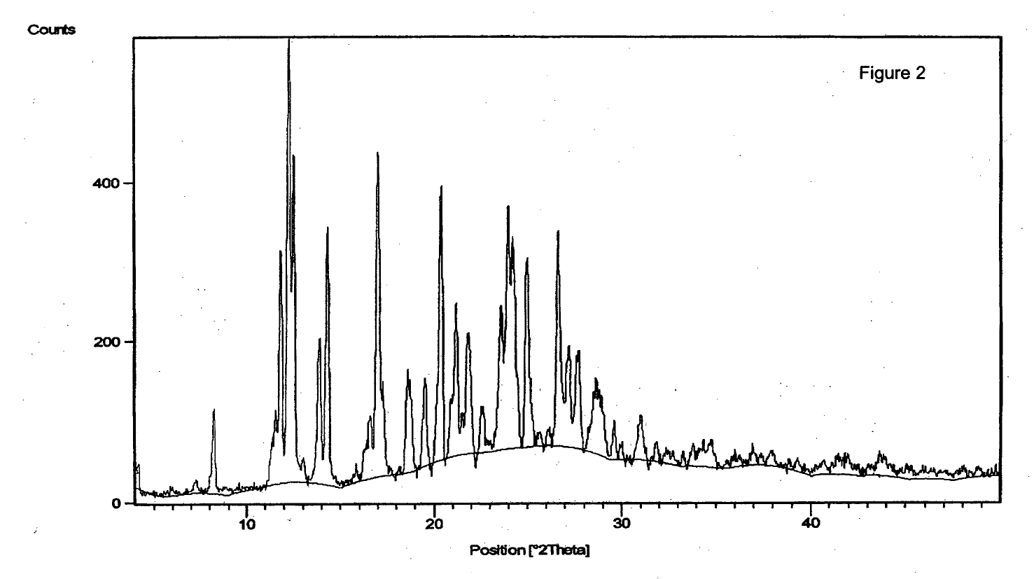

Peaks in powder X-ray diffraction pattern (See FIG. 2)

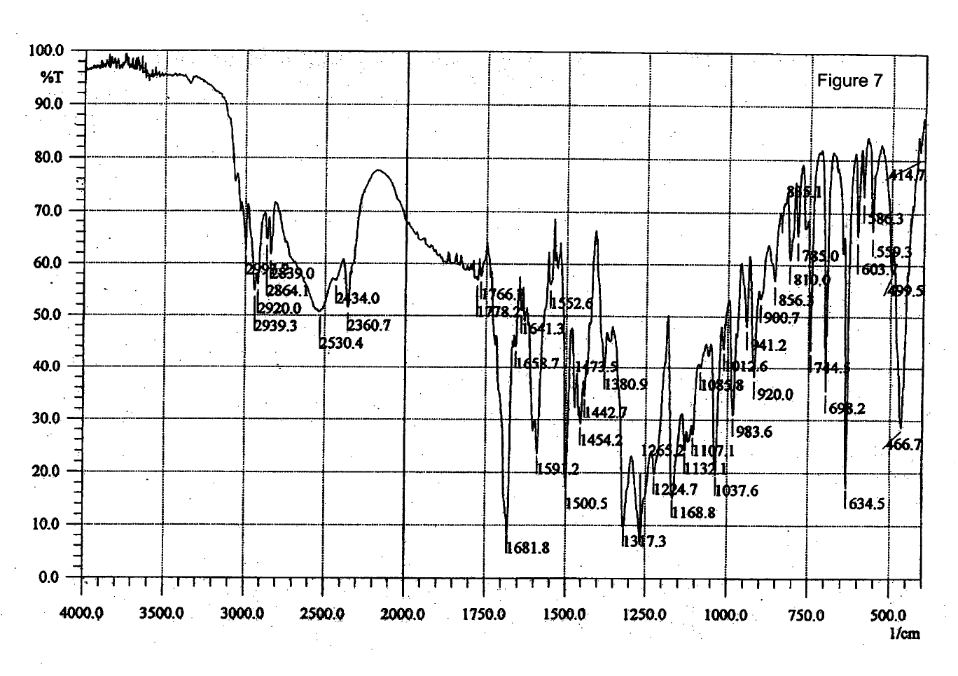

Peaks in infrared absorption spectrum in potassium bromide (See FIG. 7)

(4) Amorphous Fumarate Form

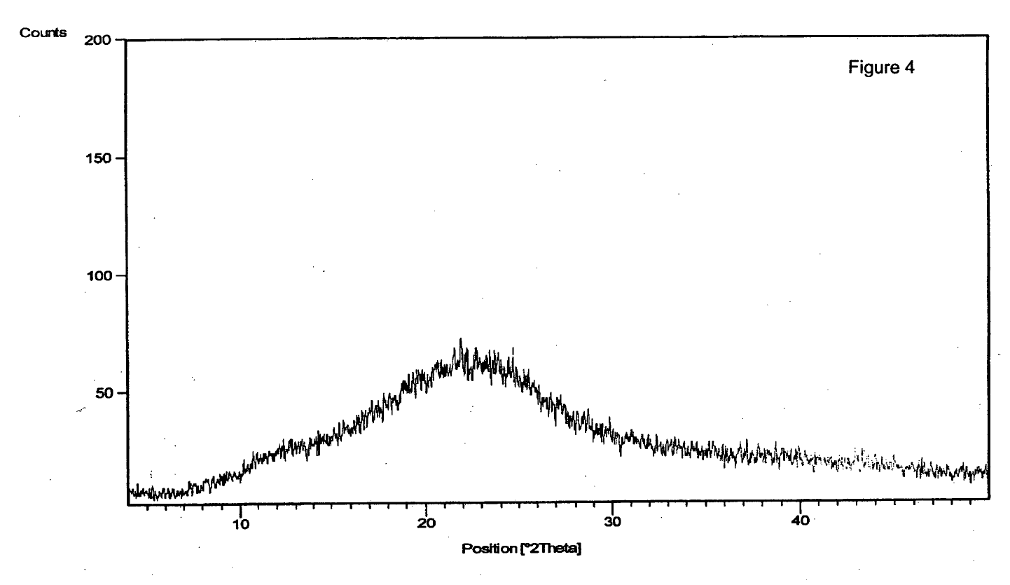

Peaks in powder X-ray diffraction pattern (See FIG. 4)

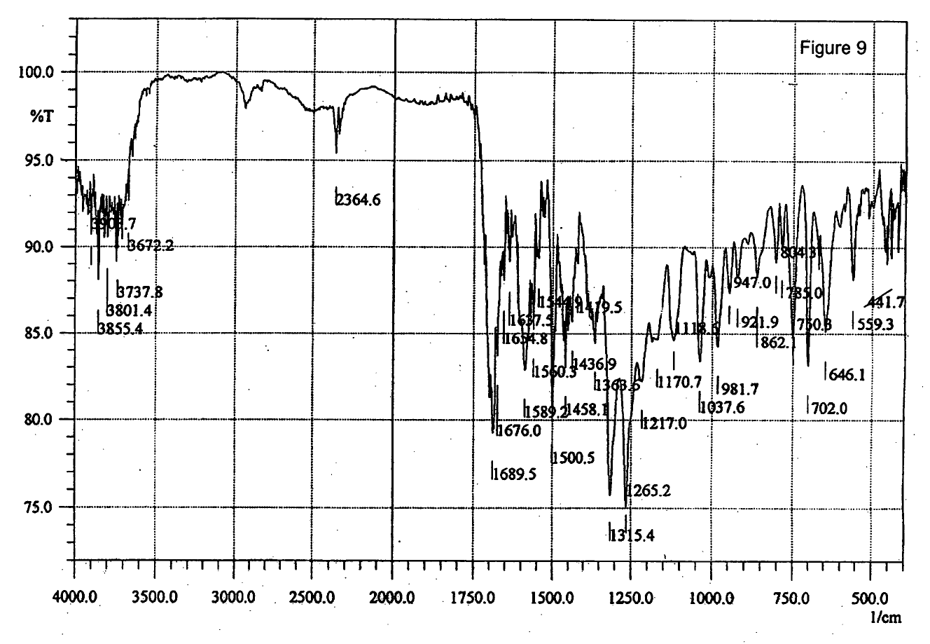

Peaks in infrared absorption spectrum in potassium bromide (See FIG. 9)

(5) Amorphous Oxalate Form

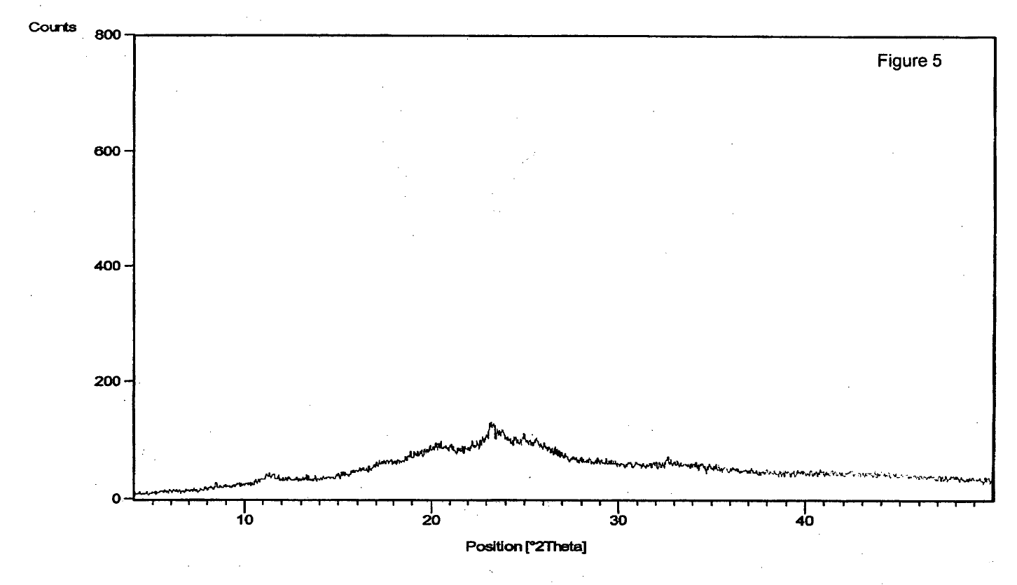

Peaks in powder X-ray diffraction pattern (See FIG. 5)

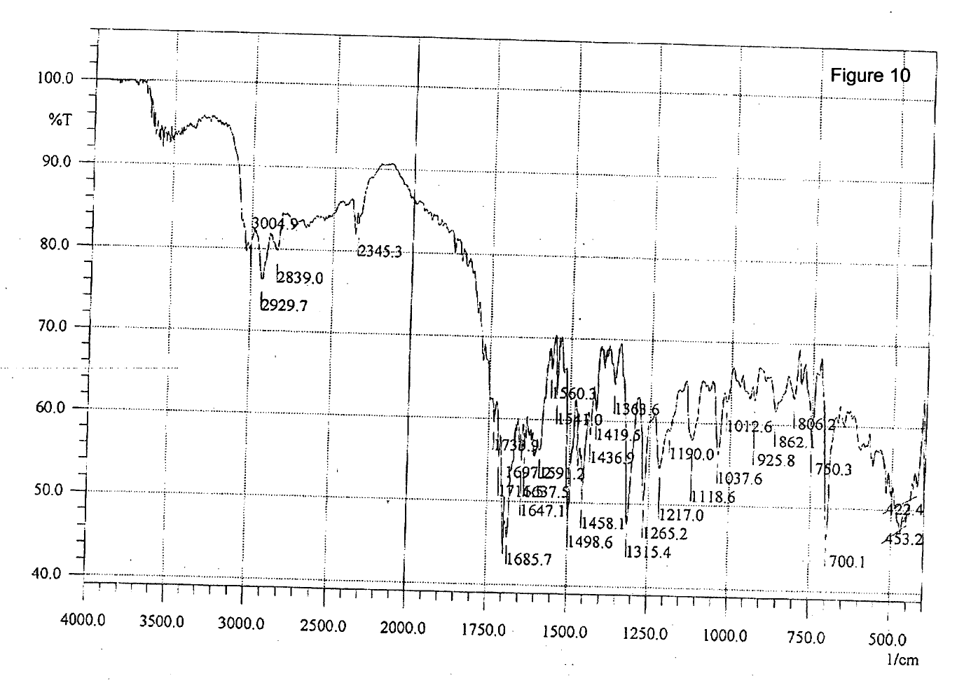

Peaks in infrared absorption spectrum in potassium bromide (See FIG. 10)

The processes for preparing novel maleate salts are given below, where in,

Donepezil base is prepared by a process as described in U.S. Pat. No. 6,649,765 B1 and is incorporated here as a reference.

(1) Maleate Form (I) Crystals:

(1-1) Process for preparing Donepezil maleate by addition of maleic acid in (1-1) acetone to Donepezil base in ethyl acetate.

(1-2) Process for preparing Donepezil maleate by addition of maleic acid in Propan-2-ol to Donepezil base in ethyl acetate.

(1-3) Process for preparing Donepezil maleate by addition of maleic acid in methanol to Donepezil base in ethyl acetate.

(1-4) Dissolving Donepezil maleate in Methanol, followed by addition of Diethyl ether.

(1-5) Dissolving Donepezil maleate in Methanol, followed by addition of Diisopropyl ether.

(1-6) Dissolving Donepezil maleate in Ethanol, followed by addition of Diisopropyl ether.

(1-7) Dissolving Donepezil maleate in Ethanol, followed by addition of Diethyl ether.

(1-8) Dissolving Donepezil maleate in Water, followed by addition of acetone.

(1-9) Dissolving Donepezil maleate in Water, followed by addition of propan-2-ol.

(1-10) Dissolving Donepezil maleate in Ethanol, followed by addition of n-hexane.

(1-11) Dissolving Donepezil maleate in Methanol and cooling to RT.

(1-12) Dissolving Donepezil maleate in Methanol, followed by addition of Toluene.

(1-13) Dissolving Donepezil maleate in water, followed by addition of Acetonitrile.

(1-14) Dissolving Donepezil maleate in Tetrahydrofuran, followed by addition of diisopropyl ether.

(1-15) The above-mentioned Donepezil maleate salt was dissolved in respective solvents by heating at 40° C.-60° C.

(2) Donepezil Maleate Amorphous Form

(2-1) Process for isolating the amorphous form which comprises spray drying, a solution of Donepezil Maleate in volatile organic solvent.

(2-2) Concentration of Donepezil maleate used for spray drying is preferably in the range of from 3 to 10% weight/volume. Spray drying is carried out in the inlet temperature range of 120° C.-200° C. and outlet temperature range 60° C.-110° C.

(2-3) Another process to obtain the amorphous form of Donepezil Maleate is evaporation of the solvent under vacuum.

(3) Fumarate Form (I) Crystals

(3-1) Process for preparing Donepezil fumarate by addition of fumaric acid in acetone to Donepezil base in ethyl acetate.

(3-2) Process for preparing Donepezil fumarate by addition of fumaric acid in Propan-2-ol to Donepezil base in ethyl acetate.

(3-3) Process for preparing Donepezil fumarate by addition of fumaric acid in methanol to Donepezil base in ethyl acetate.

(3-4) Dissolving Donepezil fumarate in Methanol, followed by addition of Diethyl ether.

(3-5) Dissolving Donepezil fumarate in Methanol, followed by addition of Diisopropyl ether.

(3-6) Dissolving Donepezil fumarate in Ethanol, followed by addition of Diisopropyl ether.

(3-7) Dissolving Donepezil fumarate in Ethanol, followed by addition of Diethyl ether.

(3-8) Dissolving Donepezil fumarate Water, followed by addition of acetone.

(3-9) Dissolving Donepezil fumarate in Water, followed by addition of propan-2-ol.

(3-10) Dissolving Donepezil fumarate in Ethanol, followed by addition of n-hexane.

(3-11) Dissolving Donepezil fumarate in Methanol and cooling to RT.

(3-12) Dissolving Donepezil fumarate in Methanol, followed by addition of Toluene.

(3-13) Dissolving Donepezil fumarate in water, followed by addition of Acetonitrile.

(3-14) Dissolving Donepezil fumarate in Tetrahydrofuran, followed by addition of diisopropyl ether.

(3-15) The above-mentioned Donepezil fumarate salt was dissolved in respective solvents by heating at 40° C.-60° C.

(4) Donepezil Fumarate Amorphous Form

(4-1) Process for isolating the amorphous form which comprises spray drying, a solution of Donepezil fumarate in volatile organic solvent.

(4-2) Concentration of Donepezil fumarate used for spray drying is preferably in the range of from 1 to 5% weight/volume. Spray drying is carried out in the inlet temperature range of 120° C.-200° C. and outlet temperature range 60° C.-110° C.

(4-3) Another process to obtain the amorphous form of Donepezil fumarate is evaporation of the solvent under vacuum

(5) Doenpezil Oxalate Amorphous Form

(5-1) Process for isolating the amorphous form which comprises spray drying, a solution of Donepezil Oxalate in volatile organic solvent.

(5-2) Concentration of Donepezil oxalate used for spray drying is preferably in the range of from 1 to 5% weight/volume. Spray drying is carried out in the inlet temperature range of 120° C.-200° C. and outlet temperature range 60° C.-110° C.

(5-3) Another process to obtain the amorphous form of Donepezil oxalate is evaporation of the solvent under vacuum.

Donepezil Maleate Form (I)

Peaks in the powder x-ray diffraction pattern are:

| | Diffraction Angle | Intensity % |
|---|---|---|
| Sr. No | (2θ°) | (I/Io) |
| 1. | 7.031 | 9.57 |
| 2. | 8.285 | 7.10 |
| 3. | 9.858 | 41.20 |
| 4. | 11.053 | 14.05 |
| 5. | 11.414 | 54.94 |
| 6. | 11.688 | 2.97 |
| 7. | 11.645 | 5.85 |
| 8. | 12.477 | 12.72 |
| 9. | 12.980 | 29.19 |
| 10. | 13.497 | 18.20 |
| 11. | 15.868 | 7.63 |
| 12. | 15.159 | 5.61 |
| 13. | 15.636 | 10.18 |
| 14. | 16.001 | 20.54 |
| 15. | 16.517 | 8.07 |
| 16. | 16.784 | 12.55 |
| 17. | 17.792 | 25.77 |
| 18. | 18.586 | 24.02 |
| 19. | 18.998 | 25.71 |
| 20. | 19.229 | 12.21 |
| 21. | 19.573 | 48.75 |
| 22. | 20.087 | 64.22 |
| 23. | 20.546 | 100.00 |
| 24. | 20.680 | 96.42 |
| 25. | 21.363 | 15.95 |
| 26. | 21.788 | 9.39 |
| 27. | 22.146 | 39.60 |
| 28. | 22.239 | 47.76 |
| 29. | 22.605 | 93.51 |
| 30. | 22.786 | 77.64 |
| 31. | 24.054 | 37.99 |
| 32. | 24.479 | 11.18 |
| 33. | 25.362 | 42.39 |
| 34. | 25.712 | 29.47 |
| 35. | 26.480 | 32.65 |
| 36. | 26.814 | 15.85 |
| 37. | 27.168 | 11.70 |
| 38. | 27.804 | 18.28 |
| 39. | 28.437 | 14.12 |
| 40. | 28.860 | 7.68 |
| 41. | 29.382 | 23.26 |
| 42. | 29.947 | 32.29 |
| 43. | 30.234 | 18.50 |
| 44. | 31.407 | 11.19 |
| 45. | 31.838 | 10.09 |
| 46. | 32.204 | 7.34 |
| 47. | 33.731 | 11.26 |
| 48. | 34.761 | 8.17 |
| 49. | 35.712 | 4.40 |
| 50. | 36.655 | 2.93 |
| 51. | 37.408 | 9.40 |
| 52. | 39.040 | 6.72 |
| 53. | 40.415 | 5.63 |
| 54. | 41.834 | 4.50 |
| 55. | 43.315 | 2.41 |
| 56. | 44.096 | 5.91 |
| 57. | 46.338 | 3.98 |

Wave numbers (cm⁻¹) of infrared absorption spectra recorded in potassium bromide are:

3375.2, 3207.4, 3016.5, 2997.2, 2947.0, 2912.3, 2723.3, 2358.8, 1697.2, 1589.2, 1569.9, 1552,6, 1500.5, 1460.0, 1355.9, 1317.3, 1303.8, 1265.2, 1218.9, 1191.9, 1161.1, 1130.2, 1105.1, 1087.8, 1066.6, 1035.7, 1010.6, 979.8, 945.1, 920.0, 898.8, 873.7, 860.2, 833.2, 806.2, 754.1, 705.9, 638.4, 605.6, 561.2, 497.6, 461.0 & 430.1

Donepezil Maleate Amorphous

Wave numbers (cm⁻¹) of infrared absorption spectra recorded in potassium bromide are:

2918, 2837.1, 1689.5, 1577.7, 1500.5, 1458.4, 1450.4, 1355.9, 1315.4, 1265.2, 1191.9, 1035.7, 945.1, 864.1, 806.1, 750.3, 702.0, 650.0, 559.3.

Fumarate Form (I)

Peaks in the powder x-ray diffraction pattern are:

| | Diffraction Angle | Intensity % |
|---|---|---|
| Sr. No | (2θ°) | (I/Io) |
| 1. | 4.210 | 6.17 |
| 2. | 8.222 | 19.58 |
| 3. | 11.887 | 44.64 |
| 4. | 12.303 | 100.00 |
| 5. | 12.558 | 65.52 |
| 6. | 13.909 | 34.31 |
| 7. | 14.367 | 58.10 |
| 8. | 17.026 | 76.00 |
| 9. | 18.591 | 20.56 |
| 10. | 19.536 | 20.22 |
| 11. | 20.433 | 60.76 |
| 12. | 21.201 | 35.43 |
| 13. | 21.795 | 27.65 |
| 14. | 22.560 | 10.17 |
| 15. | 23.596 | 33.43 |
| 16. | 24.003 | 56.55 |
| 17. | 24.285 | 43.44 |
| 18. | 25.046 | 36.93 |
| 19. | 26.645 | 50.71 |
| 20. | 27.230 | 23.42 |
| 21. | 27.721 | 22.58 |
| 22. | 28.593 | 15.28 |
| 23. | 29.573 | 8.02 |
| 24. | 31.028 | 10.24 |

Wave numbers (cm⁻¹) of infrared absorption spectra recorded in potassium bromide are:

2997.2, 2939.3, 2920.0, 2864.1, 2530.4, 2360.7, 1681.8, 1658.7, 1641.3, 1552.6, 1500.5, 1473.5, 1454.2, 1442.7, 1380.9, 1317.3, 1265.2, 1224.7, 1168.8, 1107.1, 1085.8, 1037.6, 1012.6, 983.6, 941.2, 920.0, 856.3, 810.0, 785.0, 744.5, 698.2, 634.5, 603.7, 586.3, 559.3, 449.5 and 466.7

Donepezil Fumarate Amorphous

Wave numbers (cm⁻¹) of infrared absorption spectra recorded in potassium bromide are:

1689.5, 1676.0, 1589.2, 1500.5, 1544.9, 1436.9, 1363.6, 1315.4, 1265.2, 1217.0, 1118.6, 981.7, 947, 921.9, 862.1, 804.3, 785.0, 750.3, 702.0, 646.1, 559.3

Donepezil Oxalate Amorphous

Wave numbers (cm⁻¹) of infrared absorption spectra recorded in potassium bromide are:

2929.7, 2839.0, 1685.7 1498.6, 1458.1, 1363.6, 1315.4, 1265.2, 1217.0, 1118.6, 1037.6, 862.1, 806.2, 750.3, 700.1

### EXAMPLES

Examples 1 to 13: Production of Polymorphic crystals of Donepezil maleate form (I).

Example 14 to 15: Production of amorphous form of Donepezil maleate

Examples 16 to 28: Production of Polymorphic crystals of fumarate form (I).

Example 29 to 30: Production of amorphous form of Donepezil Fumarate

Example 31 to 32: Production of amorphous form of Donepezil oxalate

The present invention will now be described in more detail with reference to the following examples. It is needless to say that the technical scope of the present invention is not limited to these examples.

### Example 1

To Donepezil base (obtained after benzylation¹) (10 gms.) in ethyl acetate (200 ml) was added maleic acid (5 gms dissolved in 100 ml acetone slowly with stirring. After addition the reaction mass was concentrated in vacuum. The solid separated was filtered and washed with acetone and dried at 60° C. afforded the title compound with a yield of 12 gms (90.2%) and melting point 176-77° C.

### Example 2

To Donepezil base (obtained after benzylation¹) (10 gms.) in ethyl acetate (200 ml) was added maleic acid (5 gms dissolved in 100 ml propan-2-ol) slowly with stirring. After addition the reaction mass was concentrated in vacuum. The solid separated was filtered and washed with acetone and dried at 60° C. afforded the title compound with a yield of 12.5 gms (96.9%) and melting point 176-78° C.

### Example 3

To Donepezil base (obtained after benzylation¹) (10 gms.) in ethyl acetate (200 ml) was added maleic acid (5 gms dissolved in 100 ml Methanol) slowly with stirring. After addition the reaction mass was concentrated in vacuum. To the residue was added 50 ml propan-2-ol. The solid separated was filtered and washed with propan-2-ol and dried at 70° C. afforded the title compound with a yield of 12 gms, 90.2%.

### Example 4

Donepezil maleate (crude) 5 gms was dissolved in Methanol 50 ml under heating at 50° C. Under stirring, at room temperature 50 ml diethyl ether was added. Stirring was continued for 1 hour after the separation of the crystals. Filtration of the crystals and drying at 60° C. afforded the title compound with a yield of 4.5 gms (90%) and melting point 177-78° C.

### Example 5

Donepezil maleate (crude) 5 gms was dissolved in Methanol 50 ml under heating at 50° C. Under stirring, at room temperature 50 ml diisopropyl ether was added. Stirring was continued for 1 hour after the separation of the crystals. Filtration of the crystals and drying at 60° C. afforded the title compound with a yield of 4.0 gms (80%) and melting point 177-78° C.

### Example 6

Donepezil maleate (crude) 5 gms was dissolved in Ethanol 50 ml under heating at 50° C. Under stirring, at room temperature 50 ml diethyl ether was added. Stirring was continued for 1 hour after the separation of the crystals. Filtration of the crystals and drying at 60° C. afforded the title compound with a yield of 4.2 gms (84%) and melting point 176-78° C.

### Example 7

Donepezil maleate (crude) 5 gms was dissolved in water 50 ml at room temperature. Under stirring, at 50° C. 100 ml acetone was added. Stirring was continued for 1 hour after the separation of the crystals. Filtration of the crystals and drying at 60° C. afforded the title compound with a yield of 4.1 gms (82%) and melting point 177-78° C.

### Example 8

Donepezil maleate (crude) 5 gms was dissolved in water 50 ml at room temperature. Under stirring, at 50° C. 100 ml propan-2-ol was added. Stirring was continued for 2 hour after the separation of the crystals at 15° C. Filtration of the crystals and drying at 60° C. afforded the title compound with a yield of 4.6 gms (92%) and melting point 176-78° C.

### Example 9

Donepezil maleate (crude) 5 gms was dissolved in ethanol 50 ml under heating at 50° C. Under stirring, at 50° C. 50 ml n-hexane was added. Stirring was continued for 1 hour after the separation of the crystals. Filtration of the crystals and drying at 60° C. afforded the title compound with a yield of 4.4 gms (88%) and melting point 177-78° C.

### Example 10

Donepezil maleate (crude) 5 gms was dissolved in methanol 25 ml under heating at 50° C. Stirring was continued for 1 hour with gradual cooling. Stirring was further continued for 1 hour after the separation of the crystals at room temperature. Filtration of the crystals and drying at 60° C. afforded the title compound with a yield of 4.0 gms (80%) and melting point 177-78° C.

### Example 11

Donepezil maleate (crude) 5 gms dissolved in methanol 50 ml under heating at 50° C. Under stirring, at 50° C. 100 ml toluene was added. Stirring was continued for 1 hour with gradual cooling. Stirring was further continued for 1 hour after the separation of the crystals. Filtration of the crystals and drying at 60° C. afforded the title compound with a yield of 4.4 gms (88%) and melting point 177-78° C.

### Example 12

Donepezil maleate (crude) 5 gms was dissolved in water 50 ml at room temperature. Under stirring, at 50° C. 100 ml acetonitrile was added. Stirring was continued for 2 hour with gradual cooling. Stirring was further continued for 1 hour after the separation of the crystals. Filtration of the crystals and drying at 60° C. afforded the title compound with a yield of 4.0 gms (80%) and melting point 176-78° C.

### Example 13

Donepezil maleate (crude) 5 gms was dissolved in tetrahydrofuran 50 ml under heating at 50° C. Under stirring, at 40° C. 100 ml diisopropyl ether was added. Stirring was continued for 1 hour after the separation of the crystals at 15° C. Filtration of the crystals and drying at 60° C. afforded the title compound with a yield of 4.2 gms (86%) and melting point 177-78° C.

### Example 15

3% weight/volume solution of Donepezil maleate in methylene dichloride is Spray dried at inlet temperature range of 120-200° C. and outlet temperature range 60-110° C. to yield amorphous Donepezil maleate (64%).

### Example 16

3% weight/volume solution of Donepezil maleate in methylene dichloride is evaporated under vacuum at temperature range of 50-70° C. to yield amorphous Donepezil maleate (72%).

### Example 17

To Donepezil base (obtained after benzylation¹) (10 gms.) in ethyl acetate (200 ml) was added maleic acid (5 gms dissolved in 100 ml propan-2-ol) slowly with stirring. After addition the reaction mass was concentrated in vacuum. The solid separated was filtered and washed with acetone and dried at 60° C. afforded the title compound with a yield of 12.3 gms (95.4%) and melting point 172-75° C.

### Example 18

To Donepezil base (obtained after benzylation¹) (10 gms) in ethyl acetate (200 ml) was added fumaric acid (5 gms dissolved in 100 ml Methanol) slowly with stirring. After addition the reaction mass was concentrated in vacuum. To the residue was added 50 ml propan-2-ol. The solid separated was filtered and washed with propan-2-ol and dried at 70° C. afforded the title compound with a yield of 12.2 gms 92%.

### Example 19

Donepezil fumarate (crude) 5 gms was dissolved in Methanol 50 ml under heating at 50° C. Under stirring, at room temperature 50 ml diethyl ether was added. Stirring was continued for 1 hour after the separation of the crystals. Filtration of the crystals and drying at 60° C. afforded the title compound with a yield of 4.55 gms (91%) and melting point 172-75° C.

### Example 20

Donepezil fumarate (crude) 5 gms was dissolved in Methanol 50 ml under heating at 50° C. Under stirring, at room temperature 50 ml diisopropyl ether was added. Stirring was continued for 1 hour after the separation of the crystals. Filtration of the crystals and drying at 60° C. afforded the title compound with a yield of 4.2 gms (84%) and melting point 172-75° C.

### Example 21

Donepezil fumarate (crude) 5 gms was dissolved in Ethanol 50 ml under heating at 50° C. Under stirring, at room temperature 50 ml diethyl ether was added. Stirring was continued for 1 hour after the separation of the crystals. Filtration of the crystals and drying at 60° C. afforded the title compound with a yield of 4.1 gms (82%) and melting point 172-75° C.

### Example 22

Donepezil fumarate (crude) 5 gms was dissolved in water 50 ml at room temperature. Under stirring, at 50° C. 100 ml acetone was added. Stirring was continued for 1 hour after the separation of the crystals. Filtration of the crystals and drying at 60° C. afforded the title compound with a yield of 4.25 gms (85%) and melting point 172-75° C.

### Example 23

Donepezil maleate (crude) 5 gms was dissolved in water 50 ml at room temperature. Under stirring, at 50° C. 100 ml propan-2-ol was added. Stirring was continued for 2 hour after the separation of the crystals at 15° C. Filtration of the crystals and drying at 60° C. afforded the title compound with a yield of 4.5 gms (90%) and melting point 172-75° C.

### Example 24

Donepezil fumarate (crude) 5 gms was dissolved in water 50 ml under heating at 50° C. Under stirring, at 50° C. 50 ml n-hexane was added. Stirring was continued for 1 hour after the separation of the crystals. Filtration of the crystals and drying at 60° C. afforded the title compound with a yield of 4.5 gms (90%) and melting point 172-75° C.

### Example 25

Donepezil fumarate (crude) 5 gms was dissolved in methanol 25 ml under heating at 50° C. Stirring was continued for 1 hour with gradual cooling. Stirring was further continued for 1 hour after the separation of the crystals at room temperature. Filtration of the crystals and drying at 60° C. afforded the title compound with a yield of 4.3 gms (86%) and melting point 172-75° C.

### Example 26

Donepezil fumarate (crude) 5 gms was dissolved in methanol 50 ml under heating at 50° C. Under stirring, at 50° C. 100 ml toluene was added. Stirring was continued for 1 hour with gradual cooling. Stirring was further continued for 1 hour after the separation of the crystals. Filtration of the crystals and drying at 60° C. afforded the title compound with a yield of 4.3 gms (86%) and melting point 172-75° C.

### Example 27

Donepezil fumarate (crude) 5 gms was dissolved in water 50 ml at room temperature. Under stirring, at 50° C. 100 ml acetonitrile was added. Stirring was continued for 2 hour with gradual cooling. Stirring was further continued for 1 hour after the separation of the crystals. Filtration of the crystals and drying at 60° C. afforded the title compound with a yield of 4.1 gms (82%) and melting point 176-78° C.

### Example 28

Donepezil fumarate (crude) 5 gms was dissolved in water 50 ml under heating at 50° C. Under stirring, at 40° C. 100 ml diisopropyl ether was added. Stirring was continued for 1 hour after the separation of the crystals at 15° C. Filtration of the crystals and drying at 60° C. afforded the title compound with a yield of 4.4 gms (88%) and melting point 172-75° C.

### Example 29

2% weight/volume solution of Donepezil Fumarate in methanol is Spray dried at inlet temperature range of 120-200° C. and outlet temperature range 60-110° C. to yield amorphous Donepezil Fumarate (66%).

### Example 30

2% weight/volume solution of Donepezil Fumarate in methanol is evaporated under vacuum at temperature range of 50-70° C. to yield amorphous Donepezil Fumarate (68%).

### Example 31

1.5% weight/volume solution of Donepezil oxalate in methylene is Spray dried at inlet temperature range of 120-200° C. and outlet temperature range 60-110° C. to yield amorphous Donepezil oxalate (73%).

### Example 32

1.5% weight/volume solution of Donepezil oxalate in methanol is evaporated under vacuum at temperature range of 50-70° C. to yield amorphous Donepezil oxalate (82%).

We claim:

1. A donepezil salt selected from the group consisting of: donepezil oxalate, donepezil maleate and donepezil fumarate.

2. The invention of claim 1, comprising donepezil maleate.

3. The invention of claim 1, comprising donepezil fumarate.

4. The invention of claim 1, comprising donepezil oxalate.

5. A process for making the invention of claim 1, said process comprising:
a. dissolving donepezil base in ethyl acetate to make a donepezil base solution;
b. dissolving a suitable acid in a second solvent to make an organic acid solution;
c. combining said donepezil base solution with said organic acid solution to make a donepezil salt solution; and
d. isolating donepezil salt from said donepezil salt solution.

6. The process of claim 5, said suitable acid comprising maleic acid.

7. The process of claim 5, said suitable acid comprising fumaric acid.

8. The process of claim 5, said isolating performed by adding an anti-solvent to said donepezil salt solution in an amount sufficient to cause said donepezil salt to form a precipitate.

9. The process of claim 8, wherein the anti-solvent comprises a compound selected from the group consisting of: tetrahydrofuran: diethyl ether; diisopropyl ether; n-hexane; propan-2-ol; acetonitrile; and toluene.

10. A process for obtaining a salt of Donepezil of amorphous crystalline structure, the process comprising evaporating a solvent from a solution containing a donepezil salt.

11. The process as claimed in claim 10 wherein the solvent comprises a lower chain alcohol and wherein said donepezil salt comprises donepezil fumarate.

12. The process as claimed in claim 11 wherein the solvent comprises a chlorinated hydrocarbon and wherein said donepezil salt is selected from the group consisting of donepezil maleate and donepezil oxalate.

13. Donepezil or a pharmaceutically acceptable salt thereof having a powder X-ray diffraction angle (2θ) selected from the group consisting of 9.858±0.4; 11.053±0.2; 11.414±0.2; 13.909±0.2; 14.367±0.4; 17.792±0.4; 20.680±0.2; and 25.712±0.4.

14. The invention of claim 13, having a powder X-ray diffraction angle (2θ) of 14.367±0.4.

15. The invention of claim 14, having powder X-ray diffraction angles (2θ) of 14.367±0.4 and 13.909±0.2.

16. The invention of claim 13, having a powder X-ray diffraction angle of (2θ) of 9.858±0.4.

17. The invention of claim 13, having a powder X-ray diffraction angle of (2θ) of 17.792±0.4.

18. The invention of claim 13, having a powder X-ray diffraction angle of (2θ) of 25.712±0.4.

19. The invention of claim 13, having a powder X-ray diffraction angle of (2θ) of 9.858±0.4, 17.792±0.4 and 25.712±0.4.

20. The invention of claim 13, having at least three powder X-ray diffraction angles (2θ) selected from the group consisting of 9.858±0.4; 11.053±0.2; 11.414±0.2; 17.792±0.4; 20.680±0.2; and 25.712±0.4.
